(19) United States
(12) Patent Application Publication
(10) Pub. No.: US 2011/0105803 A1
(43) Pub. Date: May 5, 2011

(54) METHOD OF SYNTHESIZING SEVOFLURANE

(75) Inventor: Zhiquan Zhao, Linyi (CN)

(73) Assignee: Lunan Pharmaceutical Group Corporation

(21) Appl. No.: 13/001,796

(22) PCT Filed: Jul. 2, 2009
(86) PCT No.: PCT/CN2009/000750

§ 371 Date: Dec. 29, 2010

(30) Foreign Application Priority Data
Jul. 2, 2008 (CN) 200810129596.X

(51) Int. Cl. C07C 43/12

(52) U.S. Cl. 568/683

(57) ABSTRACT

The present invention provides a method of synthesizing sevoflurane, which comprises the following steps: taking hexafluoro isopropanol as the starting material and reacting it with trioxymethylene (or paraformaldehyde) in the presence of acid to generate dihexafluoro isopropanol formal derivatives, adding anhydrous aluminum trihalide to generate halomethyl 2,2,2-trifluoro-1-(trifluoromethyl)ethyl ether, then reacting the halomethyl compound with metal fluoride to form the sevoflurane. The method is of low cost, and the reaction condition is easy to implement, and produces sevoflurane in large scale.

### FIELD OF THE INVENTION

The present invention relates to a method of synthesizing fluoromethyl-2,2,2-trifluoro-1-(trifluoromethyl)ethyl ether (known as sevoflurane).

### BACKGROUND OF THE INVENTION

In recent years, it has been reported that fluorinated ethers exhibit potent inhaled anesthetic properties. Such kind of anesthetics includes desflurane (CF₃CHFOCHF₂), isoflurane (CF₃CHClOCHF₂), enflurane (ClFCHCF₂OCHF₂) and sevoflurane ((CF₃)₂CHOCH₂F). Due to the short induction period and wake up period of sevoflurane, i.e. the desired characteristics for being an inhaled anesthetic, sevoflurane has been used as a very excellent inhaled anesthetic.

The U.S. Pat. Nos. 3,683,092 and 3,689,571 have disclosed the use of sevoflurane as an inhaled anesthetic, and also a method of synthesizing sevoflurane, which involves reacting chloromethyl 2,2,2-trifluoro-1-(trifluoromethyl)ethyl ether with an excess amount of potassium fluoride in a high-boiling point solvent at 120° C. to substitute chloromethyl with fluorine. These patents have also disclosed a synthesizing method, which involves reacting hexafluoro isopropanol and dimethyl sulfate with sodium hydroxide solution, followed by fluorinating the thus obtained methyl 2,2,2-trifluoro-1-(trifluoromethyl)ethyl ether using bromine trifluoride to produce sevoflurane. The U.S. Pat. No. 4,328,376 has disclosed a method of separating sevoflurane from the olefinic side products produced by a method similar to that disclosed in U.S. Pat. No. 3,689,571.

Other synthetic routes for sevoflurane can be found in the following publications: U.S. Pat. No. 3,897,502, which involves fluorinating the methyl 2,2,2-trifluoro-1-(trifluoromethyl)ethyl ether using argon that contains 20% fluorine; U.S. Pat. Nos. 4,250,334 and 4,469,898, which involves chloromethylating hexafluoro isopropanol using hydrogen fluoride, formaldehyde and sulfuric acid or other dehydrating agents; and PTC international application WO 97/25303, which involves the reaction between hexafluoro isopropanol and di(fluoromethyl)ether.

Okazaki et. al. has disclosed a method that involves an electrochemical fluorination for yielding fluoromethyl ether (Fluorine Chemistry, 1974, 4(4), 387). The German Patent 25 20 962 has disclosed a method of synthesizing fluoromethyl ether from chloromethyl ether and hydrogen fluoride at 125° C.-149° C. in the presence of chromium oxyfluoride. Bensoam et. al. [Tetrahedron Lett., 1979, 4, 353] has reported a method of synthesizing fluoromethyl ether by undergoing halogen exchange with tetrahydroxyfluoro phosphorane. The German Patent 2823 969 has disclosed a method of preparing organofluoride (including monofluoromethyl ether) from the reaction between the corresponding organochloride or bromide and a specifically selected ammonium hydrogenfluoride. Triethylamine hydrogenfluoride and pyridine hydrogenfluoride represent the typical examples of fluorinating agent for the preparation of said kind of organofluoroide, typically with the yield of about 40˜80% for fluorides. The Chinese patent 1244187 has made an improvement on this process with better result.

In addition, The U.S. Pat. No. 4,874,901 has reported the reaction of chloromethyl 2,2,2-trifluoro-1-(trifluoromethyl)ethyl ether with pure potassium fluoride at high temperature and high pressure, of which, however, the reaction conversion is low.

The U.S. Pat. No. 6,100,434 has reported a method of preparing sevoflurane using hexafluoro isopropanol as the staring material, in which chloromethyl 2,2,2-trifluoro-1-(trifluoromethyl)ethyl ether is firstly formed from hexafluoro isopropanol in the presence of anhydrous aluminum trichloride and trioxymethylene, and then chloromethyl 2,2,2-trifluoro-1-(trifluoromethyl)ethyl ether is reacted with a metal fluoride to substitute chlorine of chloromethyl with fluorine. Said method, however, has the following technical problems: (1) the first step of the reaction is a three-phase reaction, solidification occurs during the reaction, which makes stirring difficult, and readily leads to a bumping phenomenon in the post-treatment process which, in turn, causes a potential safety hazard; (2) the purity of the product from the first step {chloromethyl 2,2,2-trifluoro-1-(trifluoromethyl)ethyl ether} is low and the product contains lots of impurities.

PCT international publication WO2008037039 has disclosed a method of preparing sevoflurane from an intermediate, chloromethyl 2,2,2-trifluoro-1-(trifluoromethyl)ethyl ether produced by using hexafluoro isopropanol (C₃H₂F₆O, HFIP) as the starting material, and reacting HFIP directly with a formaldehyde equivalent, a strong acid and a chlorinating agent. Said method, however, has the following technical problems: (1) low conversion of hexafluoro isopropanol, and the treatment by pH adjusting for the recovery and purification of the unreacted hexafluoro isopropanol caused the production cost increased; (2) increased amount of impurities in the final products, which makes the purification process difficult (such as P3), and purity of sevoflurane become low due to the presence of impurities.

### SUMMARY OF THE INVENTION

In view of the drawbacks of the conventional method of synthesizing sevoflurane, the present invention provides a method of synthesizing sevoflurane which overcome all these drawbacks.

The objective of the present invention is to provide a method of synthesizing sevoflurane, and in comparison with the prior art, the synthesizing method of present invention improves the production yield of sevoflurane, lowers the production cost, and simplifies the production process.

The present invention provides a method of synthesizing sevoflurane, in which hexafluoro isopropanol is used as the starting material, and reacted with trioxymethylene or paraformaldehyde in the presence of an acid to give dihexafluoro isopropanol formal derivatives; said dihexafluoro isopropanol formal derivatives are then reacted with anhydrous aluminum trihalide to form halomethyl 2,2,2-trifluoro-1-(trifluoromethyl)ethyl ether, which is then reacted with metal fluoride to give sevoflurane.

The method of synthesizing sevoflurane according to the present invention is illustrated in the following scheme:

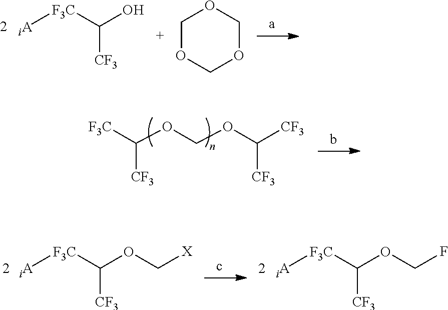

In the present invention, reaction of hexafluoro isopropanol with trioxymethylene or paraformaldehyde is conducted in an acidic environment; said acid may be a strong acid, preferably one or more acids selected from sulfuric acid, hydrochloric acid, phosphoric acid, chlorosulfonic acid, fluorosulfonic acid.

Hexafluoro isopropanol reacts with trioxymethylene or paraformaldehyde to form the dihexafluoro isopropanol formal derivatives, which are represented by the following structural formula (I):

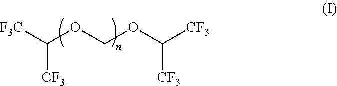

In formula (I), n is a natural number.

Typical examples of compound (I) include n=2 (i.e. dihexafluoro isopropanol diformal), n=3 (i.e. dihexafluoro isopropanol triformal), etc.

In the present invention, the main components of dihexafluoro isopropanol formal derivatives prepared from the reaction between hexafluoro isopropanol and trioxymethylene or paraformaldehyde are dihexafluoro isopropanol diformal and/or dihexafluoro isopropanol triformal.

In the present invention, reaction of dihexafluoro isopropanol formal derivatives and anhydrous aluminum trihalide can be conducted in the absence of a solvent or in the presence of a solvent. If the reaction is conducted in a solvent, the solvent is preferably selected from one or more of ethers, esters or halohydrocarbons, more preferably tetrahydrofuran, diethyl ether, ethyl acetate, chloroform or dichloromethane.

In the present invention, anhydrous aluminum trihalide used in the reaction between dihexafluoro isopropanol formal derivatives and anhydrous aluminum trihalide is anhydrous aluminum trichloride, anhydrous aluminum tribromide or anhydrous aluminum triiodide.

In the present invention, dihexafluoro isopropanol formal derivatives react with anhydrous aluminum trihalide to form halomethyl 2,2,2-trifluoro-1-(trifluoromethyl)ethyl ether of the following structural formula (II):

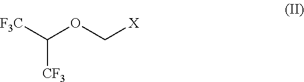

In formula (II), X is Cl, Br or I.

Typical examples of compound (II) includes X═Cl (i.e. chloromethyl 2,2,2-trifluoro-1-(trifluoromethyl)ethyl ether), X═Br (i.e. bromomethyl 2,2,2-trifluoro-1-(trifluoromethyl)ethyl ether) and X═I (i.e. iodomethyl 2,2,2-trifluoro-1-(trifluoromethyl)ethyl ether).

In the present invention, preferably, activated metal fluoride is reacted with halomethyl 2,2,2-trifluoro-1-(trifluoromethyl)ethyl ether in the presence of an activating agent. Preferably, said activating agent is ethylene glycol, diethylene glycol, triethylene glycol or 18-crown-6, and more preferably, said activating agent is triethylene glycol.

In the present invention, reaction of halomethyl 2,2,2-trifluoro-1-(trifluoromethyl)ethyl ether with metal fluoride is conducted in the absence of a solvent or in the presence of a solvent. If the reaction is conducted in the present of a solvent, the solvent is preferably an inert high-boiling point solvent, more preferably, the solvent is acetamide, sulfolane or N,N-dimethylformamide.

In the present invention, metal fluoride used in the reaction between halomethyl 2,2,2-trifluoro-1-(trifluoromethyl)ethyl ether and metal fluoride is preferably potassium fluoride, sodium fluoride or ammonium fluoride.

In one embodiment of the present invention, a method of synthesizing sevoflurane comprises the following steps:

a) hexafluoro isopropanol and trioxymethylene or paraformaldehyde solution are subjected to condensation reaction in the presence of an acid at a temperature of 0° C.˜100° C. for 4˜48 hours. After the reaction, the reaction system is left aside for phase separation to remove the acid, and then cooled to −5° C.˜5° C., crystals formed while stirring and kept the crystal formation for 1˜8 hours, and then filtered to obtain the solid retentate. The retentate is left to dry for later use. The filtrate is a solution of hexafluoro isopropanol, trioxymethylene or paraformaldehyde from incomplete conversion. After filtration, said filtrate can be used directly and repeatedly.

b) Retentate from step a) is mixed with anhydrous aluminum trihalide, and reaction is conducted at 0° C.˜60° C. for 4˜48 hours to give halomethyl 2,2,2-trifluoro-1-(trifluoromethyl)ethyl ether.

c) Activated metal fluoride is reacted with halomethyl 2,2,2-trifluoro-1-(trifluoromethyl)ethyl ether from step b) in the presence of an activating agent to give the crude sevoflurane (in accordance with the Chinese patent ZL200510071849.9, which is incorporated herein by reference in their entireties), and purified sevoflurane is obtained after distillation.

In another embodiment of the present invention, a method of synthesizing sevoflurane comprises the following steps:

a) Hexafluoro isopropanol and trioxymethylene or paraformaldehyde solution are subjected to condensation reaction in the presence of an acid at 35° C.˜45° C. for 4˜6 hours. After the reaction, the reaction system is left aside for phase separation to remove the acid, and then cooled to −5° C.˜0° C., crystal formed while stirring and kept the crystal formation for 4˜8 hours, and filtered to obtain the solid retentate. The retentate is left to dry for later use. The filtrate is a solution of hexafluoro isopropanol, trioxymethylene or paraformaldehyde from incomplete conversion. After filtration, said filtrate can be used directly and repeatedly.

b) Retentate from step a) is mixed with anhydrous aluminum trihalide, and reaction is conducted at 25° C.˜35° C. for 10˜15 hours to give halomethyl 2,2,2-trifluoro-1-(trifluoromethyl)ethyl ether.

c) Activated metal fluoride is reacted with halomethyl 2,2,2-trifluoro-1-(trifluoromethyl)ethyl ether from step b) in the presence of an activating agent to give the crude sevoflurane (in accordance with the Chinese patent ZL200510071849.9), and purified sevoflurane is obtained after distillation.

The method of preparing sevoflurane provided in the present invention involves the preparation of monohalomethyl ether from hexafluoro isopropanol via dihexafluoro isopropanol formal derivatives, from which sevoflurane is prepared. Said method is a noval method that has never been reported. Said method possesses the following advantages: (1) After the reaction, unreacted hexafluoro isopropanol and paraformaldehyde solution can be used directly and repeatedly after filtration, and no need of purification process for recovering; (2) intermediate, i.e. dihexafluoro isopropanol formal derivatives are in solid form, which facilitates the process of purification, storage and transportation, and impurities with low-boiling point can be removed easily; (3) stirring in the entire process is improved, operation become simplified, and is of lower risk; and the intermediate, i.e. halomethyl 2,2,2-trifluoro-1-(trifluoromethyl)ethyl ether is obtained with higher purity and higher production yield; (4) the method of synthesizing sevoflurane of the present invention possesses the advantages of lower consumption of starting materials, simple and controllable operation process, less three-wastes (waste solid, waste water and waste gas), lower production cost, higher conversion, readily achievable reaction condition, which is suitable for large scale industrial production.

### PREFERRED EMBODIMENTS OF THE INVENTION

By making reference to the following examples, the present invention will become more apprehended to the ordinary person in the art. However, the examples are only for illustrative purpose, but not intended to constrain the present invention in any ways.

Step 1): Preparation of the Intermediate:

### Example 1

Hexafluoro isopropanol (336 g), trioxymethylene (90 g), and concentrated sulfuric acid (15 ml) were placed in a reaction flask, stirred and heated to 40° C.˜45° C. for 6 hours, left aside for phase separation, followed by removal of the concentrated sulfuric acid, stirred and cooled to 0° C. or so, crystals formed and kept for 4 hours, and then filtered to give 149.6 g filtrate, which was then subjected to gas chromatography (GC) analysis and a mixture of hexafluoro isopropanol and trioxymethylene was confirmed. After simple filtration, said mixture was directly and repeatedly used for reaction; while the retentate obtained from the filtration was dried to give a solid of 255.5 g. Said retentate was subjected to GC analysis and confirmed as dihexafluoro isopropanol triformal (n=3) content: 41%, dihexafluoro isopropanol diformal (n=2) content: 53%, dihexafluoro isopropanol monoformal (n=1) content: <0.5%.

### Example 2

Hexafluoro isopropanol (336 g), trioxymethylene (90 g), concentrated sulfuric acid (15 ml) were placed in a reaction flask, stirred and heated to 35° C.˜40° C. for 4 hours, left aside for phase separation, followed by removal of the concentrated sulfuric acid, stirred and cooled to 0° C. or so, crystals formed and kept for 4 hours, and then filtered to give 178.8 g filtrate, which was then subjected to gas chromatography (GC) analysis and a mixture of hexafluoro isopropanol and trioxymethylene was confirmed. After simple filtration, said mixture was directly and repeatedly used for reaction, while the retentate obtained from the filtration was dried to give a solid of 250.1 g. Said retentate was subjected to GC analysis and confirmed as dihexafluoro isopropanol triformal (n=3) content: 42%, dihexafluoro isopropanol diformal (n=2) content: 52%, dihexafluoro isopropanol monoformal (n=1) content:<0.5%.

### Example 3

Hexafluoro isopropanol (336 g), paraformaldehyde (90 g), concentrated hydrochloric acid (45 ml) were placed in a reaction flask, stirred and heated to 0° C.˜5° C. for 48 hours, left aside for phase separation, followed by removal of the concentrated hydrochloric acid, stirred and cooled to −5° C. or so, crystals formed and kept for 8 hours, and then filtered to give 162.9 g filtrate, which was then subjected to gas chromatography (GC) analysis and a mixture of hexafluoro isopropanol and trioxymethylene was confirmed. After simple filtration, said mixture was directly and repeatedly used for reaction, while the retentate obtained from the filtration was dried to give a solid of 235.6 g. Said retentate was subjected to GC analysis and confirmed as dihexafluoro isopropanol triformal (n=3) content: 49%, dihexafluoro isopropanol diformal (n=2) content: 44%, dihexafluoro isopropanol monoformal (n=1) content:<0.2%.

### Example 4

Hexafluoro isopropanol (336 g), trioxymethylene (90 g), concentrated phosphoric acid (15 ml) were placed in a reaction flask, stirred and heated to 95° C.˜100° C. for 4 hours, left aside for phase separation, followed by removal of the concentrated phosphoric acid, stirred and cooled to 5° C. or so, crystals formed and kept for 1 hour, and then filtered to give 172.7 g filtrate, which was then subjected to gas chromatography (GC) analysis and a mixture of hexafluoro isopropanol and trioxymethylene was confirmed. After simple filtration, said mixture was directly and repeatedly used for reaction, while the retentate obtained from the filtration was dried to give a solid of 230.7 g. Said retentate was subjected to GC analysis and confirmed as dihexafluoro isopropanol triformal (n=3) content: 38%, dihexafluoro isopropanol diformal (n=2) content: 55%, dihexafluoro isopropanol monoformal (n=1) content: <0.9%.

Step 2): Chloromethylation of the Intermediate:

### Example 5

123.6 g solid obtained from Example 1 was weighed and placed in a reaction flask, which was then heated to 40° C.˜45° C., upon the solid was melted, reaction temperature was lowered to 20° C.˜25° C., followed by the addition of anhydrous aluminum trichloride (84.5 g) in portions, temperature was kept at 25° C.˜30° C. for 10 hours, and then cooled to 0° C. or so, which was then treated with dropwise addition of 10% diluted hydrochloric acid (500 ml), and left aside for phase separation, the organic layer was sequentially washed with 5% NaOH aqueous solution (100 ml×2), water (100 ml×2), dried over anhydrous sodium sulfate, to give 111.6 g chloromethyl 2,2,2-trifluoro-1-(trifluoromethyl)ethyl ether, purity: 98.7% (GC analysis).

### Example 6

123.6 g solid obtained from Example 1 was weighed and placed in a reaction flask, followed by the addition of 100 ml dichloromethane, and anhydrous aluminum trichloride (84.5 g) was added at 0° C. in portions, reaction temperature was kept at 30° C.˜35° C. for 15 hours, and then cooled to 0° C., which was then treated with dropwise addition of 10% diluted hydrochloric acid (530 ml), and left aside for phase separation, the organic layer was sequentially washed with 5% NaOH aqueous solution (100 ml×2), water (100 ml×2), dried over anhydrous sodium sulfate, and filtered. Filtrate was subjected to distillation to remove dichloromethane to give 113.3 g of chloromethyl 2,2,2-trifluoro-1-(trifluoromethyl)ethyl ether, purity: 98.1% (GC analysis).

### Example 7

123.6 g solid obtained from Example 1 was weighed and placed in a reaction flask, followed by the addition of 100 ml chloroform, upon the solid was dissolved, anhydrous aluminum tribromide (168.5 g) was added at 0-5° C. in portions, reaction temperature was kept at 55° C.˜60° C. for 4 hours, and then cooled to 0° C., which was then treated with dropwise addition of 10% diluted hydrobromic acid (550 ml), and left aside for phase separation, the organic layer was sequentially washed with 5% NaOH aqueous solution (100 ml×2), water (100 ml×2), dried over anhydrous sodium sulfate, chloroform was removed by distillation to give 126.6 g of bromomethyl 2,2,2-trifluoro-1-(trifluoromethyl)ethyl ether, purity: 98.8% (GC analysis).

### Example 8

123.6 g solid obtained from Example 1 was weighed and placed in a reaction flask, followed by the addition of 100 ml dichloromethane, upon the solid was dissolved, anhydrous aluminum triiodide (257.5 g) was added at 0-5° C. in portions, reaction temperature was kept at 0° C.˜5° C. for 48 hours, and then cooled to 0° C., which was then treated with dropwise addition of 10% diluted hydroiodic acid (600 ml), and left aside for phase separation, the organic layer was sequentially washed with 5% NaOH aqueous solution (100 ml×2), water (100 ml×2), dried over anhydrous sodium sulfate, dichloromethane was removed by distillation to give 146.7 g of iodomethyl 2,2,2-trifluoro-1-(trifluoromethyl)ethyl ether, purity: 98.2% (GC analysis).

Step 3): Preparation of Sevoflurane:

### Example 9

Chloromethyl 2,2,2-trifluoro-1-(trifluoromethyl)ethyl ether (500.0 g), highly active anhydrous potassium fluoride (140.0 g), acetamide (500.0 g) and triethylene glycol (60.0 g) were placed in a 1000 mL single-neck flask provided with a magnetic stirring bar and reflux apparatus, and the reaction mixture was heated to 85° C. and reflux for 6 hours. After the reaction, crude sevoflurane (388.1 g) was obtained from distillation, yield: 84.0%, purity: 97.8% (GC analysis).

Crude sevoflurane (388.1 g) was placed and subjected to distillation in a distillation apparatus to give sevoflurane of qualified grade (345.8 g), purity: 99.998% (GC analysis).

### Example 10

Bromomethyl 2,2,2-trifluoro-1-(trifluoromethyl)ethyl ether (520.0 g), highly active anhydrous potassium fluoride (130.0 g), sulfolane (500.0 g) and triethylene glycol (60.0 g) were placed in a 1000 mL single-neck flask provided with a magnetic stirring bar and reflux apparatus, and the reaction mixture was heated to 85° C. and reflux for 6 hours. After the reaction, crude sevoflurane (334.4 g) was obtained from distillation, yield: 83.6%, purity: 98.7% (GC analysis).

Crude sevoflurane (334.4 g) was placed and subjected to distillation in a distillation apparatus to give sevoflurane of qualified grade (292.9 g), purity: 99.997% (GC analysis).

### Example 11

Iodomethyl 2,2,2-trifluoro-1-(trifluoromethyl)ethyl ether (620.0 g), highly active anhydrous potassium fluoride (125.0 g), N,N-dimethylformamide (500.0 g) and triethylene glycol (60.0 g) were placed in a 1000 mL single-neck flask provided with a magnetic stirring bar and reflux apparatus, and the reaction mixture was heated to 85° C. and reflux for 6 hours. After the reaction, crude sevoflurane (331.7 g) was obtained from distillation, yield: 82.4%, purity: 98.3% (GC analysis).

Crude sevoflurane (331.7 g) was placed and subjected to distillation in a distillation apparatus to give sevoflurane of qualified grade (286.1 g), purity: 99.997% (GC analysis).

The method of synthesizing sevoflurane of the present invention has the advantages of high product quality (high purity), high production yield, ease of removal of impurities, simple purification process for recovering starting materials, low starting material consumption, which is suitable for large scale industrial production.

The present invention has been described in details in accordance with the specified embodiments as set forth. Some other modifications and equivalent variations are apparent to the ordinary person in the art and are included in the present invention.

What is claimed is:

1. A method of synthesizing sevoflurane comprising the following steps:
1) Subjecting hexafluoro isopropanol and trioxymethylene or paraformaldehyde to condensation reaction in the presence of an acid to give a solid form of dihexafluoro isopropanol formal derivatives;
2) To the thus obtained dihexafluoro isopropanol formal derivatives, adding an anhydrous aluminum trihalide to prepare the halomethyl 2,2,2-trifluoro-1-(trifluoromethyl)ethyl ether; and
3) Reacting the halomethyl 2,2,2-trifluoro-1-(trifluoromethyl)ethyl ether obtained from 2) with a metal fluoride to provide sevoflurane.

2. The method according to claim 1, wherein said acid in step 1) is one or more acids selected from sulfuric acid, hydrochloric acid, phosphoric acid, chlorosulfonic acid and fluorosulfonic acid; said metal fluoride in step 3) is metal fluoride activated in the presence of an activating agent.

3. The method according to claim 2, wherein step 1) is conducted at a temperature range from 0° C.˜100° C. for 4˜48 hours; step 2) is conducted at a temperature range from 0° C.˜60° C. for 4˜48 hours.

4. The method according to claim 3, wherein after step 1), the temperature is cooled to −5° C.˜5° C., stirred, crystals formed and kept the crystal formation for 1˜8 hours.

5. The method according to claim 1, wherein said dihexafluoro isopropanol formal derivatives obtained in step 1) are represented by the following structural formula: 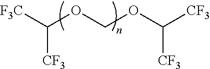
wherein, n is an integer equal to or greater than 1.

6. The method according to claim 5, wherein the main components of said dihexafluoro isopropanol formal derivatives obtained in step 1) are dihexafluoro isopropanol diformal and/or dihexafluoro isopropanol triformal.

7. The method according to claim 1, wherein in step 2), reaction of dihexafluoro isopropanol formal derivatives and anhydrous aluminum trihalide is conducted in a solvent selected from one or more of an ether, an ester and a halohydrocarbon.

8. The method according to claim 1, wherein said anhydrous aluminum trihalide in step 2) is anhydrous aluminum trichloride, anhydrous aluminum tribromide or anhydrous aluminum triiodide.

9. The method according to claim 1, wherein said halomethyl 2,2,2-trifluoro-1-(trifluoromethyl)ethyl ether obtained in step 2) is represented by the following structural formula: 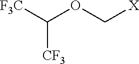
wherein, X is any one of Cl, Br or I.

10. The method according to claim 2, wherein said activating agent in step 3) is ethylene glycol, diethylene glycol, triethylene glycol or 18-crown-6.

11. The method according to claim 1, wherein step 3) is performed in an inert high-boiling point solvent.

12. The method according to claim 11, wherein said inert high-boiling point solvent is selected from acetamide, sulfolane or N,N-dimethylformamide.

13. The method according to claim 1, wherein said metal fluoride in step 3) is selected from potassium fluoride, sodium fluoride or ammonium fluoride.
